SEQUENCE LISTING (43 sequences)

SEQ ID NO: 1 - protein, 20 aa, Lactobacillus
  misc_feature (19)..(19): Xaa is Val or Ile
NTNTNAKYDVDVTPSVSAXA

SEQ ID NO: 2 - protein, 20 aa, Lactobacillus
  misc_feature (2)..(2): Xaa is Asn or Ser
GXLTGXISASYNGKXYTANL

SEQ ID NO: 3 - protein, 19 aa, Lactobacillus
  misc_feature (6)..(6): Xaa is Asn or Pro
YTVTVXDVSFNFGSENAGK

SEQ ID NO: 4 - protein, 15 aa, Lactobacillus
  misc_feature (6)..(6): Xaa is Asn or Thr
VVAAIXSKYFAAQYA

SEQ ID NO: 5 - protein, 22 aa, Lactobacillus
  misc_feature (13)..(13): Xaa is Thr or Val
HTFTVNVKATSNXNXKSATLPV

SEQ ID NO: 6 - protein, 16 aa, Lactobacillus
  misc_feature (12)..(12): Xaa is Ala or Pro
VTVPNVAEPTVXSVSK

SEQ ID NO: 7 - protein, 444 aa, Lactobacillus acidophilus
MKKNLRIVSAAAAALLAVAPVAASAVSTVSAATTINASSSAINTNTNAKYDVDVTPSVSAVAANTANNTPAIAGNLTGTISASYNGKTYTANLKADTENATITAAGSTTAVKPAELAAGVAYTVTVNDVSFNFGSENAGKTVTLGSANSNVKFTGTNSDNQTETNVSTLKVKLDQNGVASLTNVSIANVYAINTTDNSNVNFYDVTSGATVTNGAVSVNADNQGQVNVANVVAAINSKYFAAQYADKKLNTRTANTEDAIKAALKDQKIDVNSVGYFKAPHTFTVNVKATSNTNGKSATLPVVVTVPNVAEPTVASVSKRIMHNAYYYDKDAKRVGTDSVKRYNSVSVLPNTTTINGKTYYQVVENGKAVDKYINAANIDGTKRTLKHNAYVYASSKKRANKVVLKKGEVVTTYGASYTFKNGQKYYKIGDNTDKTYVKVANFR

SEQ ID NO: 8 - protein, 642 aa, Mus musculus
MGIPLTWMLLVMMVTSWFTLAEASNSTEARRCSECHNNATCTVDGVVTTCSCQTGFTGDGLVCEDMDECATPWTHNCSNSSCVNTPGSFKCSCQDGFRLTPELSCTDVDECSEQGLSNCHALATCVNTEGDYLCVCPEGFTGDGWYCECSPGSCEPGLDCLPQGPDGKLVCQDPCNTYETLTEYWRSTEYGVGYSCDAGLHGWYRFTGQGGVRMAETCVPVLRCNTAAPMWLNGSHPSSSEGIVSRTACAHWSDQCCRWSTEIQVKACPGGFYIYNLTAPPECNLAYCTDPSSVEGTCEECRVDEDCISDNGRWRCQCKQDSNITDVSQLEYRLECGANDIKMSLRKCQLQSLGFMNVFMYLNDRQCSGFSESDERDWMSIVTPARNGPCGTVLRRNETHATYSNTLYLANAIIIRDIIIRMNFECSYPLDMKVSLKTSLQPMVSALNISLGGTGKFTVRMALFQSPTYTQPHQGPSVMLSTEAFLYVGTMLDGGDLSRFVLLMTNCYATPSSNSTDPVKYFIIQDSCPRTEDTTIQVTENGESSQARFSVQMFRFAGNYDLVYLHCEVYLCDSTSEQCKPTCSGTRFRSGNFIDQTRVLNLGPITRQGVQASVSKAASSNLRLLSIWLLLFPSATLIFMVQ

SEQ ID NO: 9 - DNA, 1929 bp, Mus musculus
  CDS (1)..(1929)
atggggatccctttgacctggatgctgctggtaatgatggtaacctcctggttcactctggctgaagccagtaactcaacagaagcgagacggtgttctgaatgccacaacaacgccacctgcacggtggatggtgtggtcacaacgtgctcctgccagaccggcttcactggtgatgggctggtgtgtgaggacatggatgagtgtgctaccccatggactcacaactgctccaacagcagctgtgtgaacaccccgggctcgtttaagtgctcctgtcaggatggttttcgtctgacgcctgagctgagctgcactgatgtggatgagtgctcagagcaggggctcagtaactgtcatgccctggccacctgtgtcaacacagaaggcgactacttgtgcgtgtgtcccgagggctttacaggggatggttggtactgtgagtgctccccaggctcctgtgagccaggactggactgcttgccccagggcccggatggaaagctggtgtgtcaagacccctgcaatacatatgagaccctgactgagtactggcgcagcacagagtatggtgtgggctactcctgtgacgcgggtctgcacggctggtaccggttcacaggccagggtggcgttcgcatggctgagacctgtgtgcccgtcctgcgatgcaacacggcggcacccatgtggctcaatggctctcatccctcgagtagtgaaggcattgtgagccgcacggcctgtgcacactggagcgaccaatgctgccggtggtccacagagatccaggtgaaggcttgcccaggtggcttctatatttacaacttgacagcgccccctgagtgcaatctggcttactgcaccgatcctagttccgtggaggggacttgcgaagaatgcagggtagatgaagattgcatatcggataacggcagatggcgctgccagtgtaaacaggactccaacatcacagatgtctcccaattggagtacaggctggagtgtggggccaatgacatcaagatgtccctcagaaagtgccagctacagagtttgggctttatgaatgtcttcatgtacctgaatgacagacaatgctcaggcttcagtgagagtgatgaacgagactggatgtccatagtgacccctgccaggaatggtccctgtgggacagtattgaggagaaacgaaacccatgccacctacagcaacaccctctacctggcaaatgcgatcatcattcgggacatcatcataagaatgaactttgaatgctcttaccctctggacatgaaagtcagcctgaagacctccctacagcccatggtcagtgccctgaacatcagcttgggtgggacaggcaagttcaccgtgcggatggcattgttccagagccctacctacacacagccccaccaaggtccttctgtgatgctgtccactgaggcttttctgtatgtgggcaccatgctggatgggggtgacttgtcccggtttgtactgctaatgaccaactgctatgccacacccagtagcaactccacagaccctgtgaaatacttcattatccaggacagttgtccacgtacagaagatacaaccattcaggtgacagagaatggcgagtcatctcaggcccgattttctgttcagatgttccggtttgcaggaaactacgaccttgtctaccttcactgcgaggtgtacctatgtgactctacgagtgaacagtgtaaacctacctgctctggtactagatttcgaagtgggaacttcatagatcagacccgtgtcctgaacttgggtcccataacacgacaaggtgtccaggcctcagtgtccaaggctgcttccagcaacttgaggctcctgagcatctggctgctgttgtttccctcagccactttgatcttcatggttcaatga

SEQ ID NO: 10 - DNA, 30 bp, Artificial
  Primer
cgcagatctaccatggggatccctttgacc

SEQ ID NO: 11 - DNA, 28 bp, Artificial
  Primer
cgcgtcgaccttggacactgaggcctgg

SEQ ID NO: 12 - DNA, 20 bp, Artificial
  primer
aactggaggaaggtggggat

SEQ ID NO: 13 - DNA, 19 bp, Artificial
  primer
aggaggtgatccaaccgca

SEQ ID NO: 14 - protein, 20 aa, Lactobacillus acidophilus
NTNTNAKYDVDVTPSVSAVA

SEQ ID NO: 15 - protein, 20 aa, Lactobacillus helveticus
NTNTNAKYDVDVTPSVSAIA

SEQ ID NO: 16 - protein, 20 aa, Lactobacillus crispatus
NTNANAKYDVDVTPSLTAIA

SEQ ID NO: 17 - protein, 17 aa, Lactobacillus amylovorus
DVAKYVANVNPSFTLNA

SEQ ID NO: 18 - protein, 17 aa, Artificial
  Lactobacillus gallinarum
DVTKYVANVNPSFTLNA

SEQ ID NO: 19 - protein, 20 aa, Lactobacillus acidophilus
GNLTGTISASYNGKTYTANL

SEQ ID NO: 20 - protein, 20 aa, Lactobacillus helveticus
GSLTGSISASYNGKSYTANL

SEQ ID NO: 21 - protein, 20 aa, Lactobacillus crispatus
GSLTGTISATYGGQSYTANL

SEQ ID NO: 22 - protein, 20 aa, Lactobacillus amylovorus
GSLTGSVTANVGGVTATANL

SEQ ID NO: 23 - protein, 20 aa, Lactobacillus gallinarum
GSLTGSVTANVGGVTATANL

SEQ ID NO: 24 - protein, 19 aa, Lactobacillus acidophilus
YTVTVNDVSFNFGSENAGK

SEQ ID NO: 25 - protein, 19 aa, Lactobacillus helveticus
YTVTVPDVSFNFGSENAGK

SEQ ID NO: 26 - protein, 19 aa, Lactobacillus crispatus
YTVTISGVGFNFGTANANK

SEQ ID NO: 27 - protein, 19 aa, Lactobacillus amylovorus
YSIVVNKVGFNFGANNAGK

SEQ ID NO: 28 - protein, 19 aa, Lactobacillus gallinarum
YSIVVNKVGFNFGANNAGK

SEQ ID NO: 29 - protein, 15 aa, Lactobacillus acidophilus
VVAAINSKYFAAQYA

SEQ ID NO: 30 - protein, 15 aa, Lactobacillus helveticus
VVAAITSKYFAAQYA

SEQ ID NO: 31 - protein, 15 aa, Lactobacillus crispatus
VVAAIQAKYAAAQVD

SEQ ID NO: 32 - protein, 15 aa, Lactobacillus amylovorus
ILQAIKSNFTAFQRV

SEQ ID NO: 33 - protein, 15 aa, Lactobacillus gallinarum
ILQAINSNFTAFQRI

SEQ ID NO: 34 - protein, 22 aa, Lactobacillus acidophilus
HTFTVNVKATSNTNGKSATLPV

SEQ ID NO: 35 - protein, 22 aa, Lactobacillus helveticus
HTFTVNVKATSNVNSKSATLPV

SEQ ID NO: 36 - protein, 22 aa, Lactobacillus crispatus
TSFTVNVKATSSINGLTATLPV

SEQ ID NO: 37 - protein, 22 aa, Lactobacillus amylovorus
HSFTVTVKAVSDINGKDAKLPV

SEQ ID NO: 38 - protein, 22 aa, Lactobacillus gallinarum
HSFTVTVKAVSDINGKNAELPV

SEQ ID NO: 39 - protein, 16 aa, Lactobacillus acidophilus
VTVPNVAEPTVASVSK

SEQ ID NO: 40 - protein, 16 aa, Lactobacillus helveticus
VTVPNVAEPTVPSVSK

SEQ ID NO: 41 - protein, 16 aa, Lactobacillus crispatus
VNVTNGVNTTVDSVSK

SEQ ID NO: 42 - protein, 16 aa, Lactobacillus amylovorus
FTVANVADPVVPSQTK

SEQ ID NO: 43 - protein, 16 aa, Lactobacillus gallinarum
FTVANVADPVVPSQSK

(19) United States
(12) Patent Application Publication
(10) Pub. No.: US 2017/0202908 A1
(43) Pub. Date: Jul. 20, 2017

(54) AGENT FOR PROMOTING SUBSTANCE INCORPORATION IN INTESTINAL TRACT

(71) Applicants: ASAHI GROUP HOLDINGS, LTD., Tokyo (JP); RIKEN, Wako-shi, Saitama (JP)

(72) Inventors: Sae HYAKUTAKE, Sagamihara-shi (JP); Naoyuki YAMAMOTO, Sagamihara-shi (JP); Takashi KANAYA, Wako-shi (JP); Tatsuhiko HIROTA, Sagamihara-shi (JP); Shinji FUKUDA, Wako-shi (JP); Hiroshi OHNO, Wako-shi (JP)

(21) Appl. No.: 15/313,872

(22) PCT Filed: May 21, 2015
(86) PCT No.: PCT/JP2015/064573

§ 371 Date: Nov. 23, 2016

(30) Foreign Application Priority Data
May 29, 2014 (JP) 2014-111681

(51) Int. Cl. A61K 38/16; A61K 39/00; A23L 33/135; A23K 20/10; A23L 2/52; A61K 35/747; G01N 33/569

(52) U.S. Cl. CPC A61K 38/164; A61K 35/747; A61K 39/00; G01N 33/56911; A23K 20/10; A23L 2/52; A23L 33/135; A61K 2039/541; A23V 2002/00; A23Y 2220/03

(57) ABSTRACT

An object of the present invention is to provide means for efficiently incorporating substances such as lactic acid bacteria, which are useful for intestinal immune induction, into the intestinal tract. According to the present invention, the following are provided: an agent for promoting substance incorporation in the intestinal tract which has a surface layer protein (S-layer protein) having peptide motifs found in common among lactic acid bacteria of the genus Lactobacillus; a complex of the agent for promoting substance incorporation in the intestinal tract and a substance to be incorporated into the intestinal tract; a beverage or food product or a pharmaceutical product having the complex; and a method for screening for a lactic acid bacterium having a high level of ability to be transferred into the intestinal tract, by determining the expression level of an S-layer protein in the surface layers of bacterial cells of a test lactic acid bacterium.

### TECHNICAL FIELD

The present invention relates to an agent for promoting substance incorporation in the intestinal tract, such agent comprising a surface layer protein from a lactic acid bacterium.

### BACKGROUND ART

Immune systems exist as defense mechanisms against the invasion of foreign matter from the outside world. In particular, the intestinal immune system is largest in vivo immune system, and it is composed of immunocytes and antibodies that account for 60% of the total quantity thereof in the overall immune system. In the intestinal tract, gut-associated lymphoid tissue (GALT) is formed with Peyer's patches (PPs), lamina propria (LP), lamina propria lymphocytes (LPLs), intraepithelial lymphocytes (IELs), intestinal epithelial cells (IECs), cryptopatches (CPs), and the like. The intestinal tract is the largest in vivo immune organ. In particular, microfold cells (M cells) present in follicle associated epithelium (FAE) that covers the luminal faces of Peyer's patches are cells specialized for the uptake of antigens containing pathological microorganisms. M cells transfer antigens to various immunocompetent cells such as dendritic cells, thereby inducing the subsequent immunoresponse. It is therefore expected that it will become possible to efficiently promote immunocyte activity by targeting M cells, which serve as gates for antigen uptake.

To date, methods using ligands for effective antigen delivery to M cells, such as a Yersinia-derived invasin and a reovirus-derived σ1 protein that function when a pathogen invades M cells, have been proposed (Non-Patent Documents 1 to 3). However, concern remains about safety of components from materials like pathogens, which are not suitable for edible use, and in vivo consumption of such materials is problematic. For such reason, there is demand for a delivery system that targets M cells and uses components from highly safe materials for edible use.

Meanwhile, lactic acid bacteria are microorganisms (probiotics) that regulate the intestinal environment and act on the intestinal immune system. Lactic acid bacteria have been used as functional components of foods consumed on a daily basis, such as lactic acid bacteria beverages and yogurt. Probiotic lactic acid bacteria are also presumed to be incorporated into the intestinal tract via M cells on Peyer's patches so as to act on Toll-like receptor (TLR) or Nod-like receptor (NLR) present in dendritic cells under M cells (i.e., inside Peyer's patches), thereby inducing various forms of immunoresponse such as T cell activation, proliferation of intestinal epithelial cells, promotion of IgA production, suppression of inflammation, and the like. Therefore, it is essential to establish means for efficiently incorporating probiotic lactic acid bacteria for the use of such bacteria.

### PRIOR ART DOCUMENTS

### Non-Patent Documents

[Non-Patent Document 1] Hussan N., Florence A. T., Pharm. Res., 15, 153-156 (1998)
[Non-Patent Document 2] Wu Y., Wang X., Csencsits K. L., Haddad A., Walters N., Pascual D. W., Proc. Natl. Acad. Sci. U.S.A., 98, 9318-9323 (2001)
[Non-Patent Document 3] Wang X., Hone D. M., Haddad A., Shata M. T., Pascual D. W., J. immunol., 171, 4717-4725 (2003)

### SUMMARY OF INVENTION

### Problems to be Solved by the Invention

An object of the present invention is to provide means for efficiently incorporating substances such as lactic acid bacteria, which are useful for intestinal immune induction, into the intestinal tract.

### Means for Solving the Problem

As a result of intensive studies to achieve the above object, the present inventors found that the surface layer protein (e.g., SlpA protein) of the Lactobacillus acidophilus strain L-92 is involved in promotion of the binding of the lactic acid bacterial strain to a uromodulin protein (Umod) expressed in M cells and incorporation of the lactic acid bacterial strain via M cells. Further, the present inventors examined the number of fluorescent beads to which SlpA protein was bound that were incorporated into Peyer's patches. As a result, it was confirmed that more beads with SlpA protein had been incorporated than beads without SlpA protein. This resulted in the finding that SlpA protein can be used as a novel delivery molecule for the intestinal tract. Furthermore, the present inventors found that it is possible to readily conduct in vitro screening for lactic acid bacteria having excellent ability to be incorporated into the intestinal tract using, as an index, the expression or non-expression of a protein having peptide motifs found in common among lactic acid bacteria of the genus Lactobacillus, such peptide motifs being present in the amino acid sequence of SlpA protein in the bacterial cell surface layers of lactic acid bacteria. The above findings have led to the completion of the present invention.

Specifically, the present invention encompasses the following inventions.
(1) An agent for promoting substance incorporation in the intestinal tract, comprising a surface layer protein of a lactic acid bacterium of the genus Lactobacillus that comprises at least one of the peptide motifs consisting of amino acid sequences of the following formulae (I) to (VI) and has activity of binding to uromodulin (Umod) protein, or a fragment of the surface layer protein:

formula (I):
Asn-Thr-Asn-Thr-Asn-Ala-Lys-Tyr-Asp-Val-Asp-Val-
Thr-Pro-Ser-Val-Ser-Ala-X1-Ala (where X1
represents Val or Ile);
formula (II):
Gly-X2-Leu-Thr-Gly-X3-Ile-Ser-Ala-Ser-Tyr-Asn-
Gly-Lys-X4-Tyr-Thr-Ala-Asn-Leu (where X2
represents Asn or Ser, X3 represents Thr or
Ser, and X4 represents Thr or Ser);
formula (III):
Tyr-Thr-Val-Thr-Val-X5-Asp-Val-Ser-Phe-Asn-Phe-
Gly-Ser-Glu-Asn-Ala-Gly-Lys (where X5
represents Asn or Pro);
formula (IV):
Val-Val-Ala-Ala-Ile-X6-Ser-Lys-Tyr-Phe-Ala-Ala-
Gln-Tyr-Ala (where X6 represents Asn or Thr);
formula (V):
His-Thr-Phe-Thr-Val-Asn-Val-Lys-Ala-Thr-Ser-Asn-
X7-Asn-X8-Lys-Ser-Ala-Thr-Leu-Pro-Val(where X7
represents Thr or Val and X8 represents Gly or
Ser);
and
formula (VI):
Val-Thr-Val-Pro-Asn-Val-Ala-Glu-Pro-Thr-Val-X9-
Ser-Val-Ser-Lys (where X9 represents Ala or Pro).

(2) The agent for promoting substance incorporation in the intestinal tract according to (1), comprising a surface layer protein of a lactic acid bacterium of the genus Lactobacillus that comprises at least one peptide motif consisting of an amino acid sequence including a deletion, substitution, or addition of 1 to 10 amino acids with respect to any one of the amino acid sequences of formulae (I) to (VI) and has activity of binding to uromodulin (Umod) protein.
(3) The agent for promoting substance incorporation in the intestinal tract according to (1) or (2), wherein the surface layer protein is an S-layer protein from Lactobacillus acidophilus, Lactobacillus helveticus, Lactobacillus crispatus, Lactobacillus amylovorus, or Lactobacillus gallinarum.
(4) The agent for promoting substance incorporation in the intestinal tract according to any one of (1) to (3), wherein the surface layer protein is SlpA protein from Lactobacillus acidophilus.
(5) The agent for promoting substance incorporation in the intestinal tract according to (4), wherein SlpA protein from Lactobacillus acidophilus is a protein selected from the group consisting of the following (a) to (c):

(a) a protein consisting of the amino acid sequence of SEQ ID NO: 7;

(b) a protein consisting of an amino acid sequence including a deletion, substitution, or addition of one to several amino acids with respect to the amino acid sequence of SEQ ID NO: 7; and

(c) a protein having a sequence identity of 90% or more with the amino acid sequence of SEQ ID NO: 7.
(6) A complex of the agent for promoting substance incorporation in the intestinal tract according to any one of (1) to (5) and a substance to be incorporated into the intestinal tract.
(7) The complex according to (6), wherein the substance to be incorporated into the intestinal tract is a food component.
(8) The complex according to (7), wherein the food component is a lactic acid bacterium.
(9) The complex according to (6), wherein the substance to be incorporated into the intestinal tract is a pharmaceutical component.
(10) The complex according to (9), wherein the pharmaceutical component is a mucosal vaccine antigen.
(11) A composition comprising the complex according to any one of (6) to (10).
(12) The composition according to (11), which is a beverage or food product, a pharmaceutical product, or an animal feed.
(13) A method for screening for a lactic acid bacterium having a high level of ability to be transferred into the intestinal tract, comprising determining the level of expression of a protein comprising at least one of the peptide motifs consisting of the amino acid sequences of the following formulae (I) to (VI) in the surface layers of bacterial cells of a test lactic acid bacterium:

formula (I):
Asn-Thr-Asn-Thr-Asn-Ala-Lys-Tyr-Asp-Val-Asp-Val-
Thr-Pro-Ser-Val-Ser-Ala-X1-Ala (where X1
represents Val or Ile);
formula (II):
Gly-X2-Leu-Thr-Gly-X3-Ile-Ser-Ala-Ser-Tyr-Asn-
Gly-Lys-X4-Tyr-Thr-Ala-Asn-Leu (where X2
represents Asn or Ser, X3 represents Thr or
Ser, and X4 represents Thr or Ser);
formula (III):
Tyr-Thr-Val-Thr-Val-X5-Asp-Val-Ser-Phe-Asn-Phe-
Gly-Ser-Glu-Asn-Ala-Gly-Lys (where X5
represents Asn or Pro);
formula (IV):
Val-Val-Ala-Ala-Ile-X6-Ser-Lys-Tyr-Phe-Ala-Ala-
Gln-Tyr-Ala (where X6 represents Asn or Thr);
formula (V):
His-Thr-Phe-Thr-Val-Asn-Val-Lys-Ala-Thr-Ser-Asn-
X7-Asn-X8-Lys-Ser-Ala-Thr-Leu-Pro-Val(where X7
represents Thr or Val and X8 represents Gly or
Ser);
and
formula (VI):
Val-Thr-Val-Pro-Asn-Val-Ala-Glu-Pro-Thr-Val-X9-
Ser-Val-Ser-Lys (where X9 represents Ala or Pro).

This patent application claims priority from Japanese Patent Application No. 2014-111681 filed on May 29, 2014, and it includes part or all of the contents as disclosed in the descriptions thereof.

### Effects of the Invention

A surface layer protein (S-layer protein) from a lactic acid bacterium that is an active ingredient for the agent for promoting substance incorporation in the intestinal tract of the present invention has activity of binding to intestinal tract M cells and activity of promoting substance incorporation via the intestinal tract M cells. Therefore, the agent for promoting substance incorporation in the intestinal tract of the present invention can promote quantitative increase of a useful lactic acid bacterium or mucosal vaccine antigen incorporated into the intestinal tract, thereby acting effectively and with certainty on the intestinal immune system. This makes it possible to suppress allergic symptoms and prevent mucosal infections such as influenza. In addition, since the agent for promoting substance incorporation in the intestinal tract of the present invention comprises a protein from a lactic acid bacterium as an active ingredient, it is highly safe.

### BRIEF DESCRIPTION OF DRAWINGS

FIG. 1 shows immunostaining results for test lactic acid bacterial strains (L-92, CP23, and LiCl-L92) (SlpA is stained white).

FIG. 2 shows the Umod-binding rates of test lactic acid bacterial strains (L-92, CP23, and LiCl-L92) [Dunnet t-Test; L-92 (n=9), CP23 (n=12), and LiCl-L92 (n=12); **: p<0.01].

FIG. 3 shows the Umod-binding rates of test lactic acid bacterial strains (L-92 and L-92 treated with the anti-SlpA antibody) [Student t-Test (n=9); #: p<0.1].

FIG. 4 shows the numbers of test lactic acid bacterial strains (L-92, CP23, and LiCl-L92) incorporated via M cells [Mann-Whitney U test; L-92 vs CP23 (n=3), L-92 vs LiCl-L92 (n=4); *: p<0.05, **: p<0.01].

FIG. 5 shows the numbers of fluorescent beads (i.e., those to which SlpA was bound and those to which BSA was bound) incorporated via M cells [Student t-Test (n=4); *: p<0.05].

FIG. 6 shows multiple alignment analysis results for the S-layer proteins of Lactobacillus acidophilus (L. acidophilus), Lactobacillus helveticus (L. helveticus), and Lactobacillus gasseri (L. gasseri) (motifs 1 to 6: peptide motifs found in common between Lactobacillus acidophilus (L. acidophilus) and Lactobacillus helveticus (L. helveticus) having high levels of ability to bind to Umod but not found in Lactobacillus gasseri (L. gasseri), having a low level of ability to bind to Umod).

FIG. 7 shows structural formulae (I) to (VI) of peptide motifs found in common among the surface layer proteins (S-layer proteins) of lactic acid bacteria of the genus Lactobacillus having activity of binding to uromodulin (Umod). The formulae were determined by multiple alignment analysis.

FIG. 8 shows partial amino acid sequences of Lactobacillus acidophilus (L. acidophilus), Lactobacillus helveticus (L. helveticus), Lactobacillus crispatus (L. crispatus), Lactobacillus amylovorus (L. amylovorus), and Lactobacillus gallinarum (L. gallinarum), which correspond to amino acid sequences of peptide motifs found in common among the surface layer proteins (S-layer proteins) of lactic acid bacteria of the genus Lactobacillus having activity of binding to uromodulin (Umod) protein. (In FIG. 8, reference numerals shown above the amino acid sequences of formulae (I) to (VI) represent amino acids as follows: 1: an amino acid found in common among five bacterial species (L. acidophilus, L. helveticus, L. crispatus, L. amylovorus, and L. gallinarum); 2: an amino acid found in common among three bacterial species (L. acidophilus, L. helveticus, and L. crispatus); and 3: an amino acid found in common between two bacterial species (L. acidophilus and L. helveticus).)

### EMBODIMENTS FOR CARRYING OUT THE INVENTION

### 1. Agent for Promoting Substance Incorporation in the Intestinal Tract

The agent for promoting substance incorporation in the intestinal tract of the present invention comprises a surface layer protein of a lactic acid bacterium of the genus Lactobacillus that comprises at least one of the peptide motifs consisting of amino acid sequences of the following formulae (I) to (VI) and has activity of binding to uromodulin (Umod) protein, or a fragment of the surface layer protein:

formula (I):
(SEQ ID NO: 1)
Asn-Thr-Asn-Thr-Asn-Ala-Lys-Tyr-Asp-Val-Asp-Val-
Thr-Pro-Ser-Val-Ser-Ala-X1-Ala (where X1
represents Val or Ile);
formula (II):
(SEQ ID NO: 2)
Gly-X2-Leu-Thr-Gly-X3-Ile-Ser-Ala-Ser-Tyr-Asn-
Gly-Lys-X4-Tyr-Thr-Ala-Asn-Leu (where X2
represents Asn or Ser, X3 represents Thr or
Ser, and X4 represents Thr or Ser);
formula (III):
(SEQ ID NO: 3)
Tyr-Thr-Val-Thr-Val-X5-Asp-Val-Ser-Phe-Asn-Phe-
Gly-Ser-Glu-Asn-Ala-Gly-Lys (where X5 represents
Asn or Pro);
formula (IV):
(SEQ ID NO: 4)
Val-Val-Ala-Ala-Ile-X6-Ser-Lys-Tyr-Phe-Ala-Ala-
Gln-Tyr-Ala (where X6 represents Asn or Thr);
formula (V):
(SEQ ID NO: 5)
His-Thr-Phe-Thr-Val-Asn-Val-Lys-Ala-Thr-Ser-Asn-
X7-Asn-X8-Lys-Ser-Ala-Thr-Leu-Pro-Val (where X7
represents Thr or Val and X8 represents Gly or
Ser);
and
formula (VI):
(SEQ ID NO: 6)
Val-Thr-Val-Pro-Asn-Val-Ala-Glu-Pro-Thr-Val-X9-
Ser-Val-Ser-Lys (where X9 represents Ala or Pro).

In addition, peptide motifs contained in an S-layer protein may have mutations, as long as the protein has activity of binding to uromodulin (Umod) protein. For example, a deletion, substitution, or addition of 1 to 10 amino acids, preferably 1 to 7 amino acids, more preferably 1 to 5 amino acids, and further preferably 1 to 3 amino acids may be included, with respect to any one of the amino acid sequences of formulae (I) to (VI). Substitution of amino acids specified herein is preferably a conservative amino acid substitution, which means a substitution between amino acids having similar characteristics, such as structural and electrical characteristics and polar or hydrophobic characteristics. These characteristics can be classified based on, for example, amino acid side chain similarity. Examples of amino acids suitable for substitution include amino acids having basic side chains (lysine, arginine, and histidine), amino acids having acidic side chains (aspartic acid and glutamic acid), amino acids having aliphatic side chains (alanine, valine, leucine, isoleucine), amino acids having hydroxyl-containing side chains (serine, threonine, and tyrosine), and amino acids having amide-containing side chains (asparagine and glutamine).

Preferred examples of the S-layer protein used in the present invention are S-layer proteins from Lactobacillus acidophilus, Lactobacillus helveticus, Lactobacillus crispatus, Lactobacillus amylovorus, or Lactobacillus gallinarum.

An S-layer protein A (hereinafter referred to as “SlpA protein”) present in the surface layer of the Lactobacillus acidophilus strain L-92 is particularly preferable. SlpA protein is known as a protein having a molecular weight of 43.6 kDa and an isoelectric point of 10.4, which functions for protection, maintenance of cellular characteristics, adhesion or attachment of molecules or ions, and the like (FEMS Microbiol. Rev. 29: 511-529).

The present invention is intended to utilize the following conventionally unknown activities of SlpA protein: activity of binding to uromodulin (Umod) protein, known as a protein that is expressed in M cells present in the epithelial cell layers of Peyer's patches in the small intestine (hereinafter simply referred to as “Umod” in some cases); and activity of promoting substance incorporation via M cells. To date, it has been reported that surface layer proteins of lactic acid bacteria have ability to bind to substances. SlpA of Lactobacillus acidophilus (NCFM) binds to C-type lectin DC-SIGN (Konstantinov S R et al., S layer protein A of Lactobacillus acidophilus NCFM regulates immature dendritic cell and T cell functions. Proc Natl Acad Sci USA. 2008 Dec. 9; 105 (49):19474-9). CbsA, which is the S-layer protein of Lactobacillus crispatus (JCM 5810), binds to collagens I and V (Sillanpaa J et al., Characterization of the collagen-binding S-layer protein CbsA of Lactobacillus crispatus. J Bacteriol. 2000 November; 182(22): 6440-50). However, there have been no reports on the binding of surface layer proteins of lactic acid bacteria to proteins on intestinal tract M cells.

SlpA protein is a protein consisting of the amino acid sequence of SEQ ID NO: 7. Alternatively, SlpA protein may be a mutant of such protein as long as such mutant protein has activity of binding to Umod and activity of promoting substance incorporation via M cells, which are inherent to SlpA protein. Also, SlpA protein may be a protein having a partial sequence (i.e., a partial peptide) of the above amino acid sequence.

Examples of the mutant protein encompass a protein consisting of an amino acid sequence including a deletion, substitution, or substitution of one to several amino acids with respect to the amino acid sequence of SEQ ID NO: 7. The expression “one to several” used herein refers to a number of amino acids that can be deleted, substituted, or added by a known mutant protein production method such as site-specific mutagenesis. The number of amino acids is not limited as long as the above activities can be maintained. However, it is 1 to 30 amino acids, preferably 1 to 20 amino acids, more preferably 1 to 10 amino acids, and most preferably 1 to 5 amino acids. One example of amino acid substitution is the aforementioned conservative amino acid substitution. Further, the mutant protein may be a protein consisting of an amino acid sequence having a sequence identity of 90% or more with the amino acid sequence of SEQ ID NO: 7. The expression “sequence identity of 90% or more” used herein refers to a sequence identity of preferably 95% or higher, more preferably 97% or higher, and most preferably 98% or higher. Amino acid sequence identity can be determined by FASTA or BLAST search. The term “mutation” used herein mainly refers to a mutation that has been artificially introduced by a known mutant protein production method; however, it may refer to a similar naturally occurring mutation.

SlpA protein used in the present invention may be produced by a method of chemical synthesis based on the amino acid sequence of SEQ ID NO: 7. Alternatively, it may be produced by a gene recombination technique. When a gene recombination technique is used, it comprises producing an expression vector containing, for example, a gene encoding the amino acid sequence of SEQ ID NO: 7, transforming appropriate host cells using the expression vector so as to obtain a transformant, and culturing the transformant, thereby mass-producing a protein of interest from the obtained culture. Such expression vector can be produced and introduced into host cells by a known method.

Any cells of prokaryotic and eukaryotic organisms can be used as host cells. For example, prokaryotic cells that are generally used as host cells are cells of Escherichia coli, Bacillus subtilis, and the like. Examples of eukaryotic cells include yeast cells.

Host cells can be introduced into expression vectors by known methods such as the calcium phosphate method, liposome method, electroporation method, and particle gun method.

Known separation techniques can be used in combination for isolation and purification of proteins expressed in transformed cells. Examples of such techniques include: various chromatographic methods such as ion exchange chromatography, affinity chromatography, high performance liquid chromatography, adsorption chromatography, and gel filtration chromatography; and methods combining such chromatography and salting-out, ultrafiltration, gel filtration, dialysis, treatment with a denaturing agent such as urea or a surfactant, centrifugation, ultrasonication, and enzyme digestion.

According to the present invention, a fragment of a surface layer protein of a lactic acid bacterium of the genus Lactobacillus having activity of binding to uromodulin (Umod) protein is a fragment of the “S-layer protein” defined above. The length of such fragment is not limited as long as the fragment has activity of binding to uromodulin (Umod) protein. However, such fragment is a fragment having, for example, 20 or more amino acids, preferably 50 or more amino acids, and more preferably 100 or more amino acids of the amino acid sequence that constitutes the S-layer protein.

2. Complex of the Agent for Promoting Substance Incorporation in the Intestinal Tract and a Substance to be Incorporated into the Intestinal Tract

It is possible to efficiently incorporate a substance (hereinafter referred to as a “target substance”) into the intestinal tract by allowing the agent for promoting substance incorporation in the intestinal tract of the present invention to form a complex with the relevant target substance. Such target substance may be a substance that shows in vivo activity when incorporated into the intestinal tract. Examples thereof include food components and pharmaceutical components. In particular, food components and pharmaceutical components having the effect of inducing intestinal immunity are preferable, such as lactic acid bacteria, mucosal vaccine antigens, and the like.

Lactic acid bacteria may be viable cells or dead cells. Further, in the case of dead cells, the cells may be pulverized. Bacterial cells can be pulverized using methods and devices known in the art via, for example, physical disruption, enzymatic lysis treatment, or the like. Physical disruption can be performed by either a wet method (treatment of bacterial cell in a state of suspension) or a dry method (treatment of bacterial cell in a state of powder) involving stirring with the use of a homogenizer, a ball mill, a bead mill, Dyno-Mill, a planetary mill, or the like, pressurization with the use of a jet mill, a French press, a cell crusher, or the like, or filtration. Enzymatic lysis treatment allows disruption of cell walls of bacterial cells with the use of enzymes such as lysozyme.

The agent for promoting substance incorporation into the intestinal tract may be covalently or non-covalently bound to a target substance in the complex of the present invention. Means for covalent or non-covalent binding is not particularly limited.

For example, if the target substance is a lactic acid bacterium that lacks an S-layer protein in the surface layers of its bacterial cells, a complex can be formed by allowing the agent for promoting substance incorporation in the intestinal tract to bind directly or via an appropriate cross-linking agent (linker) to the lactic acid bacterial cell surface layer. An example of a method for producing such complex is a method using a protein cross-linking agent, which targets, for example, an amino group, a carboxyl group, or a sulfhydryl group, and preferably an amino group, of a protein (e.g., a sugar-chain receptor protein) that is present in the surface layers of the lactic acid bacterial cells to be incorporated, and which has a reactive functional group capable of binding to such group. Examples of a protein cross-linking agent that may be used include commercially available products such as glutaraldehyde, EDAC (1-ethyl-3-(3-dimetylaminopropyl) carbodimide, hydrochloride), DSP (dithiobis(succinimidyl propionate)), and DCC (N,N′-dicyclohexylcarbodiimide).

Alternatively, the following methods can be used: a method for allowing anchor motifs (e.g., CWBD motif, LysM motif, and GW motif) that are involved in non-covalent binding between lactic acid bacterial cells and cell walls to ligate to an S-layer protein so as to immobilize the protein in the surface layers of lactic acid bacterial cells; a method allowing an anchor motif (e.g., LPXTG motif) that is involved in covalent binding between lactic acid bacterial cells and cell walls to ligate to an S-layer protein so as to cause forced expression of the protein; and a method for forming a peptide bond between any protein localized in the surface layers of lactic acid bacterial cells and an S-layer protein using a protein cross-linking enzyme (transglutaminase).

In addition, if a target substance is a mucosal vaccine antigen, it is possible to allow the agent for promoting substance incorporation in the intestinal tract to bind directly or via an appropriate carrier to a mucosal vaccine antigen, thereby forming a complex. Specifically, it is possible to employ a method for producing a fusion protein of a mucosal vaccine antigen to be incorporated and an S-layer protein, a method for allowing a mucosal vaccine antigen encapsulated in a carrier into which a reaction group has been introduced to bind to an S-layer protein, or the like. Examples of carriers include liposomes, microspheres or nanospheres, biodegradable carriers such as Poly (lactic-co-glycolic) acid (PLGA), and mucosa-adhering carriers comprising hyaluronic acid, chitin, or the like.

A fusion protein of an S-layer protein and a mucosal vaccine antigen can be produced by a known gene recombination technique. For example, it is possible to obtain such fusion protein by artificially ligating a gene encoding an S-layer protein and a gene encoding a mucosal vaccine antigen protein to prepare a fusion gene, inserting the fusion gene downstream of a promoter of an expression vector, and transfecting appropriate host cells, thereby causing the fusion gene to be expressed. The order of binding an S-layer protein and a mucosal vaccine antigen is not limited for such fusion protein. In addition, information on the base sequences of genes encoding mucosal vaccine antigen proteins can be obtained from known databases (e.g., GenBank). It is also possible to obtain nucleotide sequence information via cloning of genes of interest and nucleotide sequence analysis by known methods.

Further, when, for example, liposome is used as the aforementioned carrier, the lipid composition or size of liposome and the method of binding an S-layer protein to liposome are not particularly limited. For preparation of liposome, in addition to phosphatidylcholine, cholesterol, polyethylene glycol-bound lipid, or the like, a lipid having a lipid-terminal carboxyl group or maleimide group is used for binding of an S-layer protein. Encapsulation of antigens into liposomes can be carried out by a known freezing and thawing method, a reverse phase evaporation method, a hydration method, or the like. The S-layer protein is added to the prepared liposome encapsulating an antigen so as to produce a conjugate of the liposomes and the S-layer protein in a peptide condensation reaction system or an SH-group reaction system.

3. Composition Comprising the Complex of the Agent for Promoting Substance Incorporation in the Intestinal Tract and a Substance to be Incorporated into the Intestinal Tract

The above complex can be added to a composition such as a beverage or food product, a pharmaceutical product, or an animal feed together with an appropriate additive. The term “pharmaceutical product” used herein encompasses pharmaceutical products for animals as well as pharmaceutical products for humans. The term “animal feed” also refers to feeds for livestock (e.g., pigs and cattle) and pet foods for pet animals (e.g., dogs and cats).

A complex containing a lactic acid bacterium as a substance to be incorporated into the intestinal tract can be added to a beverage or food product. According to the present invention, the term “beverage or food product” refers to a health food or beverage, a functional food or beverage, a nutritional supplement, or a food or beverage for specified health use. Most appropriate examples of beverage or food products include dairy products such as yogurt, cheese, and beverages containing lactic acid bacteria, and pickles. A beverage or food product may be in any form suitable for edible use, such as in solid, liquid, granule, grain, powder, capsule (hard or soft capsule), cream, or paste form. In particular, examples of forms suitable for health foods and functional foods include tablets, capsules, granules, and powders. For example, a health food in the tablet form can be produced by preparing a product that is prescribed to contain a lactic acid bacterium to which the agent for promoting substance incorporation in the intestinal tract of the present invention has been bound by the above method and compressing a such product into a predetermined shape, kneading such product using water or a solvent such as alcohol so as to form a wet product in a predetermined shape, or introducing such product into a predetermined mold for molding.

A complex containing a mucosal vaccine antigen as the substance to be incorporated into the intestinal tract can be mixed with a pharmaceutical product and, in particular, a mucosal vaccine preparation. In such case, it may be mixed together with an adjuvant to enhance immune response. Examples of an adjuvant include aluminum hydroxide, BCG, aluminum phosphate, keyhole limpet hemocyanin, dinitrophenol, dextran, and TLR ligands (e.g., lipopolysaccharide (LPS) and CpG).

The mucosal vaccine antigen is not particularly limited as long as it can induce mucosal immune response. However, it is typically an antigen from a pathogen of mucosal infection. Such pathogen of mucosal infection may be a virus or a bacterium. Examples of viruses include, but are not limited to, influenza virus, human immunodeficiency virus (HIV), chickenpox virus, measles virus, rubella virus, poliovirus, rotavirus, adenovirus, herpes virus, and severe acute respiratory syndrome (SARS) virus. In addition, examples of bacteria include, but are not limited to, Bordetella pertussis, Neisseria meningitidis, Haemophilus influenzae Type b, pneumococcus, and Mycobacterium tuberculosis. Antigens from these pathogens may be from natural products or they may be produced by an artificial method involving gene recombination or the like.

The above vaccine antigen also includes allergens used for hyposensitization therapy. The term “allergen vaccine” refers to a vaccine that is administered in vivo as an allergen to produce an IgG antibody against the allergen, thereby blocking the action of IgE that cause allergies, or to increase allergen-specific type 1 helper T cells (Th1 cells) in vivo, thereby reducing type 2 helper T cells involved in allergic symptoms (Th2 cells). It is possible to suppress allergic reactions by causing hyposensitization using such vaccine. Examples of allergens include, but are not limited to, food allergens (casein, lactalbumin, lactoglobulin, ovomucoid, ovalbumin, conalbumin, etc.), house dust allergens (mite allergens, etc.), pollen allergens (cedar pollen allergen, ragweed allergen, orchard grass allergen, etc.), and allergens such as animal hair and the like.

4. Method for Screening for a Lactic Acid Bacterium Having a High Level of Ability to be Transferred into the Intestinal Tract

According to the present invention, a method for screening for a lactic acid bacterium having a high level of ability to be transferred into the intestinal tract is also provided. The method comprises determining the expression level of an S-layer protein in the surface layers of bacterial cells of a test lactic acid bacterium based on the Umod-binding activity of the SlpA protein and activity of promoting substance incorporation via M cells.

The S-layer protein may be used directly or labeled with an arbitrary labeling substance before use. Examples of labeling substances include fluorescent substances, radioactive isotopes (¹²⁵I, ³H, ¹⁴C, ³⁵S, etc.), chemiluminescent substances, biotin, marker proteins, and peptide tags. Examples of marker proteins include the Fc region of an antibody, alkaline phosphatase, and horse radish peroxidase (HRP). Examples of peptide tags include FLAG, 6× His or 10× His comprising 6 or 10 His (histidine) residues, and fragments of influenza hemagglutinin (HA).

According to the present invention, the term “ability to be transferred into the intestinal tract” refers to the ability to adhere to intestinal tract M cells so as to transit into M cells and then reach inside the internal portions of Peyer's patches via the M cell basement membrane.

In the screening method of the present invention, the expression level of the S-layer protein in the bacterial cell surface layers of a test lactic acid bacterium may be obtained through determination of the absolute S-layer protein amount by, for example, comparing the test lactic acid bacterium with a standard sample. However, it is not always necessary to perform quantification of the absolute S-layer protein amount. Evaluation is considered to be sufficient if it enables clarification of the relative relationship between the S-layer protein in the bacterial cell surface layers of a test lactic acid bacterium and that of a control lactic acid bacterium.

The S-layer protein expression level can be determined by a known protein expression analysis method. A typical example of such method is immunoassay using an antibody against an S-layer protein. Examples of immunoassay that can be employed include, but are not particularly limited to, conventionally known methods such as enzyme immunoassay (EIA), latex agglutination, immunochromatography, Western blotting, radioimmunoassay (RIA), fluorescence immunoassay (FIA), luminescence immunoassay, spin immunoassay, a turbidimetric method for determining turbidity associated with antigen-antibody complex formation, an enzyme sensor electrode method for detecting the potential change due to the binding of an antigen to an antibody-bound solid membrane electrode, and immunoelectrophoresis. Of these, EIA and Western blotting are preferable. In addition, EIA encompasses competitive enzyme immunoassay, sandwich enzyme-linked immunosorbent solid phase assay (sandwich ELISA), and the like.

An antibody against an S-layer protein used for the above determination can be obtained using a method known to those skilled in the art. Such antibody may be a polyclonal antibody or a monoclonal antibody. In addition, an active fragment of an antibody may be used as such antibody. Examples of an active fragment include F(ab′)2, Fab′, Fab, and Fv. For example, a polyclonal antibody against an S-layer protein is obtained by collecting blood from a mammal (e.g., a rabbit, rat, or mouse) sensitized with an antigen and separating serum from the blood by a known method. A serum containing a polyclonal antibody may be used instead of a polyclonal antibody. Further, in order to obtain a monoclonal antibody, antibody-producing cells (e.g., spleen cells and lymph node cells) are removed from the above mammal sensitized with an antigen and fused with cells such as myeloma cells. The thus obtained hybridoma is cloned. An antibody can be collected from the resulting culture and designated as a monoclonal antibody.

For detection of an S-layer protein, the above antibodies can be labeled according to need. Examples of a labeling substance that can be used include the enzymes described above, radioisotopes, and fluorochromes. In addition, it is possible to label a substance that specifically binds to an antibody, such as protein A or protein G, without labeling the antibody, thereby indirectly detecting the antibody.

A test lactic acid bacterium may be of any lactic acid bacterial strain belonging to the genus Lactobacillus, Lactococcus, Bifidobacterium, Leuconostoc, Streptococcus, Enterococcus, Pediococcus, Weissella, Oenococcus, or the like. Examples of lactic acid bacteria belonging to the genus Lactobacillus include Lactobacillus acidophilus, Lactobacillus brevis, Lactobacillus casei, Lactobacillus delbrueckii, Lactobacillus fermentum, Lactobacillus helveticus, Lactobacillus kefir, Lactobacillus paracasei, Lactobacillus plantarum, Lactobacillus bulgaricus, Lactobacillus rhamnosus, Lactobacillus salivarius, Lactobacillus johnsonii, Lactobacillus gasseri, Lactobacillus amylovorus, Lactobacillus crispatus, and Lactobacillus gallinarum. Examples of lactic acid bacteria belonging to the genus Lactococcus include Lactococcus lactis, Lactococcus plantarum, and Lactococcus raffinolactis. Examples of lactic acid bacteria belonging to the genus Bifidobacterium include Bifidobacterium infantis, Bifidobacterium breve, Bifidobacterium longum, Bifidobacterium pseudolongum, Bifidobacterium bifidum, Bifidobacterium animalis, Bifidobacterium adolescentis, Bifidobacterium catenulatum, and Bifidobacterium pseudocatenulatum. Examples of lactic acid bacteria belonging to the genus Leuconostoc include Leuconostoc lactis and Leuconostoc mesenteroides. Examples of lactic acid bacteria belonging to the genus Streptococcus include Streptococcus thermophilus and Streptococcus lactis. Examples of lactic acid bacteria belonging to the genus Enterococcus include Enterococcus faecalis, Enterococcus durance, and Enterococcus faecium. Examples of lactic acid bacteria belonging to the genus Pediococcus include Pediococcus pentosaceus. Examples of lactic acid bacteria belonging to the genus Weissella include Weissella cibaria, Weissella confusa, and Weissella halotolerans. Examples of lactic acid bacteria belonging to the genus Oenococcus include Oenococcus oeni.

### EXAMPLES

The present invention is described in more detail below with reference to the Examples; however, the present invention is not limited to these Examples.

### Example 1

### Quantitative Determination of SlpA in Lactic Acid Bacterial Strains

### (1) Preparation of Lactic Acid Bacterial Strains

The Lactobacillus acidophilus strain L-92 and the Lactobacillus acidophilus strain CP23 were used in this experiment. Each bacterial strain was statically cultured using an MRS medium (Difco) at 37° C. for 20 hours and then washed three times with PBS. Each resultant was suspended in PBS.

LiCl treatment of the L-92 was carried out by washing the L-92 twice with PBS, removing the obtained supernatant, and statically incubating the resultant for a certain period of time in a solution of 5 M LiCl (Wako) at room temperature. After incubation, the resultant was washed again twice with PBS and then resuspended in PBS.

### (2) Immunostaining

Each bacterial cell suspension (10 μl) was applied to a microscope slide, dried, and heat-fixed using an alcohol lamp. Mouse anti-SlpA (clone 383) (1.4 mg/ml) was diluted 100-fold with PBS and added to the microscope slide. A reaction was allowed to take place at room temperature for 2 to 3 hours. Following this, each microscope slide was washed three times with PBS. Further, Cy3-streptavidin (Cy3-conjugated Streptavidin, ImmunoResearch Laboratories Inc., No. 016-160-084) was diluted 200-fold with PBS and added to the microscope slides. A reaction was allowed to take place at room temperature for 2 to 3 hours. Then, each microscope slide was washed three times with PBS.

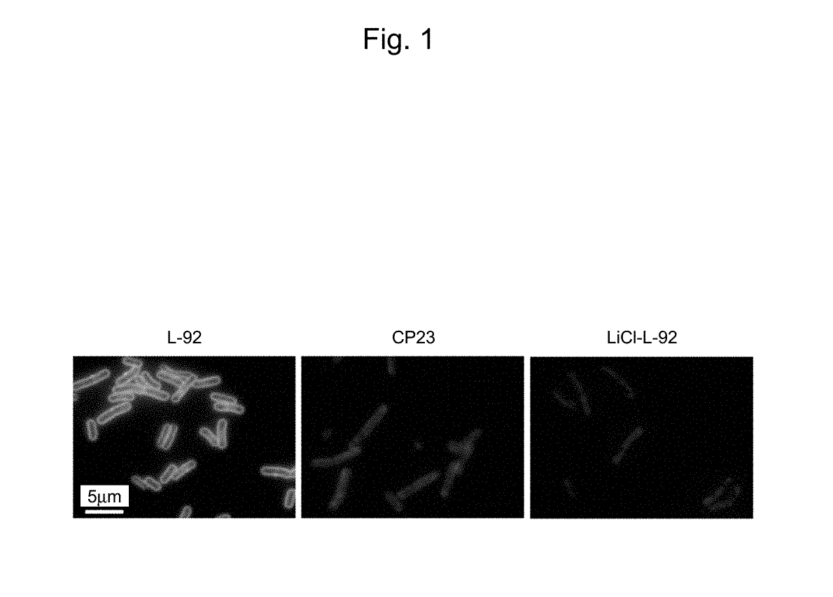

After having been enclosed with a coverslip, the bacterial cells of each strain were observed using a fluorescent microscope in order to visually confirm fluorescence intensity. FIG. 1 shows the results.

The results showed that the bacterial cell surfaces of the L-92 had been covered with SlpA, while on the other hand, SlpA had been slightly localized on the bacterial cell surfaces of the CP23. The results also showed that SlpA had been almost completely removed from the bacterial cell surfaces of the L-92 that had been treated with LiCl (FIG. 1).

### Example 2

### Evaluation of the Degree of Binding to Umod for Lactic Acid Bacterial Strains

### (1) Preparation of Lactic Acid Bacterial Strains

The L-92, the CP23, and the LiCl-treated L-92, which had been prepared as specified in Example 1, were used in this experiment. Anti-SlpA antibody treatment of the L-92 was carried out by suspending the L-92 in PBS, in which the anti-SlpA antibody was dissolved so as to result in a final concentration of 140 μg/ml, and the resulting solution was gently shaken at 4° C. overnight.

### (2) Umod Binding Test (In Vitro Binding Assay)

The L-92, the CP23, the LiCl-treated L-92 (LiCl-L92), and the L-92 treated with the anti-SlpA antibody were examined in terms of the degree of binding to Umod.

First, a fusion protein (Fc-mUmod), which is expressed by ligating the mouse Umod protein (corresponding to positions 1-616 of SEQ ID NO: 8) to the Fc domain of human IgG1, was prepared according to Hase K. et al., Uptake through glycoprotein 2 of FimH1 bacteria by M cells initiates mucosal immune response, Nature 2009, 462: 226-31. The following primers were used for amplifying the mUmod (mouse Umod) sequence (SEQ ID NO: 9): Forward primer: 5′-CGCAGATCTACCATGGGGATCCCTTTGACC-3′ (SEQ ID NO: 10); and Reverse primer: 5′-CGCGTCGACCTTGGACACTGAGGCCTGG-3′ (SEQ ID NO: 11). The fusion protein was cloned into a pcDNA3 vector (Invitrogen) into which an Fc domain had been inserted using restriction enzymes (BglII and SalI).

The vector into which Fc-mUmod had been cloned was introduced into human embryonic kidney cells (HEK293T cells) and the cells were cultured for 7-10 days. The Fc-mUmod protein secreted in the resulting supernatant was collected and purified using an HiTrap protein AHP affinity column (GE Healthcare).

Next, the Fc-mUmod protein and hIgG as a control Fc protein were respectively diluted with PBS so as to result in a concentration of 5 μg/ml, and the resultants were applied to a 96-well plate (50 μl per well) and immobilized at 4° C. overnight. Each well was washed three times with 200 μl of PBS. Then, a 1% BSA/PBS solution (200 μl) was applied thereto for blocking at room temperature for 2 hours. Then, the blocking solution was removed. Thereafter, bacterial cells of each test lactic acid bacterium, which had been suspended in PBS so as to result in a concentration of 10⁶ cells/50 μl, were applied thereto (50 μl per well) and incubated at room temperature for 2 hours. Each well was washed five times with 200 μl of PBS and then PBS was completely removed.

DNA was extracted from bacterial cells bound to each plate using NucleoSpin™ Tissue (Takara) in accordance with the provided protocol. Real-time PCR was performed using the extracted DNA as a template and universal primers targeting the 16S rRNA gene (F: 5′-AACTGGAGGAAGGTGGGGAT-3′ (SEQ ID NO: 12), R: 5′-AGGAGGTGATCCAACCGCA-3′ (SEQ ID NO: 13)). The number of bacterial cells bound to Fc-mUmod and the number of bacterial cells bound to hIgG were quantitatively determined in accordance with the protocol provided with SYBR™ Premix Ex Taq™ II (Tli RNaseH Plus) (Takara).

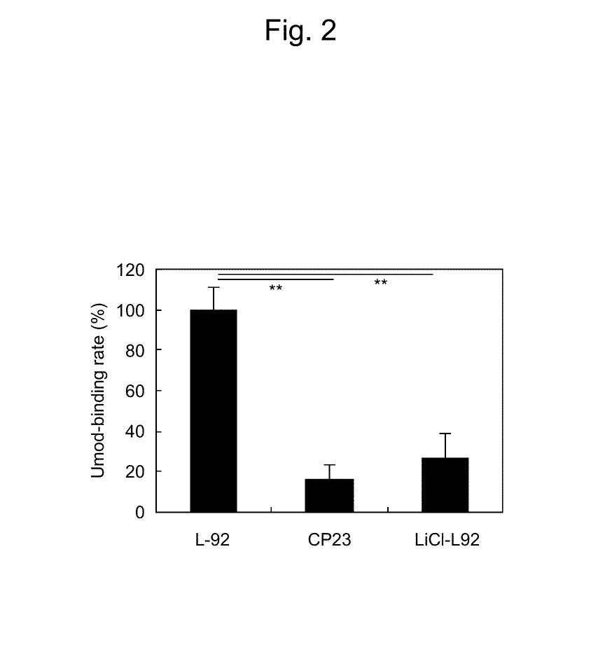

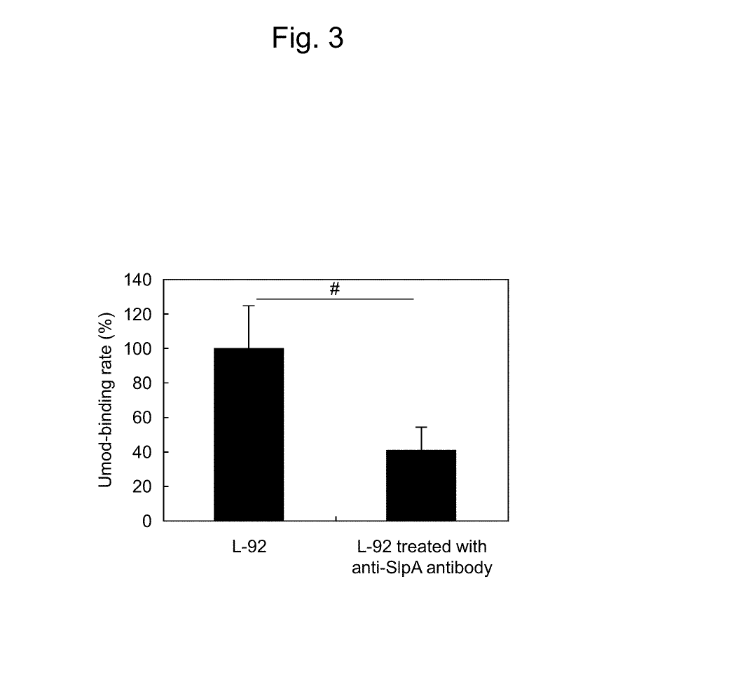

A value obtained by subtracting the number of bacterial cells bound to hIgG from the number of bacterial cells bound to Fc-mUmod was designated as the number of bacterial cells binding to Umod. The number of bacterial cells binding to Umod for the L-92 was designated as “100%,” and the numbers of bacterial cells binding to Umod for other strains were expressed as percentages relative to the number of bacterial cells binding to Umod for the L-92. FIGS. 2 and 3 show the results.

In the cases of the CP23 containing a small amount of the surface layer protein (SlpA) and the L-92 from which SlpA had been removed via LiCl treatment, the degree of binding to Umod was significantly lower than that in the case of the L-92, which was rich in SlpA (FIG. 2). In addition, as a result of treatment of the L-92 with the anti-SlpA antibody, the degree of binding to Umod tended to decline (FIG. 3). These results suggested that SlpA impacts the degree of binding to Umod for the L-92.

### Example 3

### Evaluation of the Numbers of Lactic Acid Bacterial Strains Uptaken by M Cells

### (1) Preparation of Lactic Acid Bacterial Strains

The L-92, the CP23, and the LiCl-treated L-92, which had been prepared as specified in Example 1, were used in this experiment.

### (2) Loop Assay

The L-92, the CP23, and the LiCl-treated L-92 were fluorescent-labeled using a Cy3 Mono-Reactive Dye Pack (GE Healthcare) and suspended in PBS so as to result in a concentration of 10⁹ cells/ml. Fluorescent labeling was performed in accordance with the protocol recommended by the manufacturer. C57BL/6J mice were fasted for several hours and subjected to laparotomy under isoflurane anesthesia. Both ends of the Peyer's patch region of the small intestine were ligated with a suture to form a loop, and 100 μl of a bacterial cell solution of each strain (10⁸ cells) was injected into the loop. After incubation for 1 hour, the mice were euthanized by cervical dislocation.

### (3) Immunostaining

Peyer's patches were excised, washed with 1× HBSS (GIBCO), and immobilized with BD Cytofix/Cytoperm™ (BD Bioscience). The resultant was sufficiently washed with a wash solution, which had been prepared by 1-fold (1×) dilution of Perm/Wash™ Buffer 10× (BD Bioscience) with the use of milliQ water and the addition of Saponin from Quillaja bark (SIGMA) so as to result in a final concentration of 0.1%. Then, blocking was performed using a blocking solution prepared by suspending BSA in such wash solution so as to result in a concentration of 0.2%.

The resultant was incubated in a primary antibody solution, which had been prepared by 100-fold dilution of Rat anti-mouse GP2 IgG2a (clone 2F11-C3) (1.0 mg/ml) with a blocking solution, and washed with a wash solution. Next, the resultant was incubated in a secondary antibody solution, which had been prepared by 200-fold dilution of Alexa Fluor™ 488 goat anti-rat IgG (H+L) (Invitrogen) (2.0 mg/ml) with a blocking solution. The obtained tissue was washed well with PBS and whole-mounted for observation.

### (4) Comparison of the Numbers of Bacterial Cells Uptaken by M Cells

The follicle associated epithelium region was photographed using a confocal microscope (Leica SP2 AOBS Conforcal and Multiphoton; Leica). Bacterial cells in a region in which by M cells immunostained with Alexa 488 and Cy3-labelled bacterial cells overlapped each other were regarded as “bacterial cells uptaken by M cells” for cell counting. The area of the portion of follicle associated epithelium was determined using Image J (downloaded at http://rsb.info.nih.gov/ij/download.html) so as to calculate the number of bacterial cells uptaken by M cells per area (Cy3⁺M cells/PP dome area (cells/mm²)).

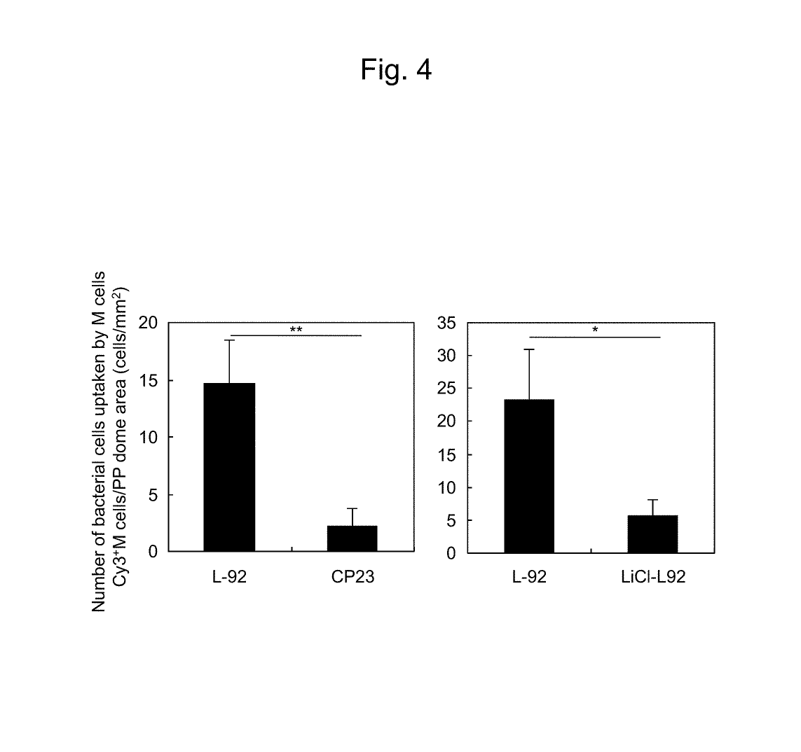

The number of bacterial cells uptaken by M cells for the L-92 was designated as “100%,” and the numbers of bacterial cells uptaken by M cells for other strains were expressed as percentages relative to the number of bacterial cells uptaken by M cells for the L-92. FIG. 4 shows the results. The CP23 and the LiCl-L92 characterized by a low degree of binding to Umod were significantly inferior to the L-92 in terms of the degree of uptake by M cells. Accordingly, it was suggested that SlpA is involved in uptake of the L-92 by M cells.

### Example 4

### Quantitative Evaluation of SlpA-Bound Carriers Uptaken by M Cells

### (1) Isolation and Purification of SlpA

The Lactobacillus acidophilus strain L-92 was incubated in 5M LiCl for 30 minutes so as to extract SlpA. The resulting bacterial cells were removed by centrifugation, followed by dialysis with a PBS solution using a dialysis tube ( 20/32 inch, Nihon Medical Science, Inc.). Thus, SlpA was obtained. As a result of SDS-PAGE and CBB staining, it was confirmed that a single band had been obtained.

### (2) Binding of Proteins to Fluorescent Beads

SlpA that had been isolated and purified in (1) and BSA were each allowed to covalently bind to fluorescent beads of two different colors (FluoSpheres (registered trademark) calboxylate-modified microspheres, 1.0 mm, orange/yellow-green (Invitrogen)) using EDAC [1-Ethyl-3-(3-dimetylaminopropyl) carbodimide, hydrochloride] (Dojindo Laboratories) in accordance with the protocols recommended by the manufacturers.

### (3) Loop Assay

Loop assay was performed as desctibed in Example 3, except that the incubation time was set to 2 hours.

(4) Comparison of Numbers of Beads Incorporated into Peyer's Patches

Peyer's patches were excised and washed with 1× HBSS (GIBCO). Then, a frozen block was prepared using an O.C.T. compound (Sakura Fintek USA). Frozen sections 5 mm in thickness were prepared using cryostat LEICA CM1850. The numbers of beads incorporated into Peyer's patches were determined by observation using a fluorescent microscope. Six to twelve sections were observed for each mouse so as to calculate an average.

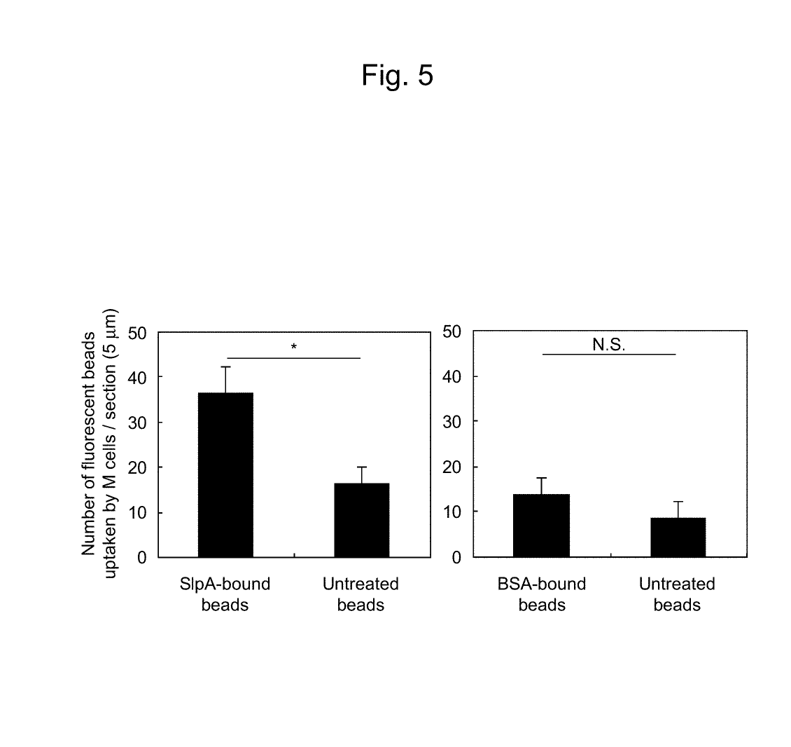

FIG. 5 shows the results of a comparison of the number of beads to which SlpA was bound that were incorporated into Peyer's patches with the number of beads to which BSA was bound that were incorporated into Peyer's patches. It was confirmed that the number of beads incorporated via M cells into Peyer's patches increases with the use of the beads to which SlpA was bound.

### Example 5

### Evaluation of the Degree of Binding to Umod for Different Lactic Acid Bacterial Strains

### (1) Preparation of Test Lactic Acid Bacteria

About 20 lactic acid bacterial strains belonging to the genus Lactobacillus were used in this experiment. Each bacterial strain was statically cultured in an MRS medium (Difco) at 37° C. for 20 hours, washed three times with PBS, and suspended in PBS as specified in Example 1.

### (2) Umod Binding Test (In Vitro Binding Assay)

Evaluation of the degree of binding to Umod for each bacterial strain was carried out as specified in Example 2.

Table 1 lists the results of calculation of the number of bacterial cells binding to Umod for 14 test lactic acid bacterial strains. A value obtained by subtracting the number of bacterial cells binding to hIgG from the number of bacterial cells bound to Fc-mUmod was designated as the number of bacterial cells binding to Umod.

TABLE 1

| | Number of bacterial |
|---|---|
| | cells binding |
| Lactic acid bacterial strain | to Umod (log₁₀) |
| Lactobacillus fermentum CP1753 | 5.4 |
| Lactobacillus fermentum CP1299 | 5.3 |
| Lactobacillus johnsonii CP1544 | 4.7 |
| Lactobacillus helveticus CP2151 | 4.3 |
| Lactobacillus delbruekii subsp. | 4.3 |
| bulgaricus CP2189 | |
| Lactobacillus delbruekii subsp. | 4.2 |
| bulgaricus CP973 | |
| Lactobacillus acidophilus L-92 | 4.1 |
| Lactobacillus acidophilus CP1613 | 4.0 |
| Lactobacillus brevis CP287 | 3.9 |
| Lactobacillus acidophilus CP734 | 3.7 |
| Lactobacillus acidophilus CP23 | 3.5 |
| Lactobacillus casei CP2517 | 3.5 |
| Lactobacillus gasseri CP793 | 3.1 |
| Lactobacillus rhamnosus CP1270 | 2.7 |

As shown in Table 1, it was confirmed that the number of bacterial cells binding to Umod varies among different bacterial species and strains of the genus Lactobacillus.

### Example 6

### Examination of Consensus Sequences Present in Amino Acid Sequences of S-Layer Proteins in the Surface Layers of Lactic Acid Bacteria

In consideration of the results in Example 5, multiple alignment analysis of S-layer proteins was carried out using ClutalW (http://clustalw.ddbj.nig.ac.jp/). Specifically, the following bacterial species of the genus Lactobacillus, the genome information of which was available, were examined: Lactobacillus acidophilus (gi|58336516|ref|YP_193101.1|S-layer protein [Lactobacillus acidophilus NCFM]) and Lactobacillus helveticus (gi|550820440|emb|CDI42266.1|Surface layer protein [Lactobacillus helveticus CIRM-BIA 953]), which had been confirmed to exhibit a relatively high degree of binding to Umod in Example 5; and Lactobacillus gasseri (gi|1619598|emb|CAA69725.1|aggregation promoting protein [Lactobacillus gasseri]), which had been confirmed to exhibit a low degree of binding to Umod in Example 5. The sequences identified herein were found in common in Lactobacillus acidophilus and Lactobacillus helveticus but not in Lactobacillus gasseri.

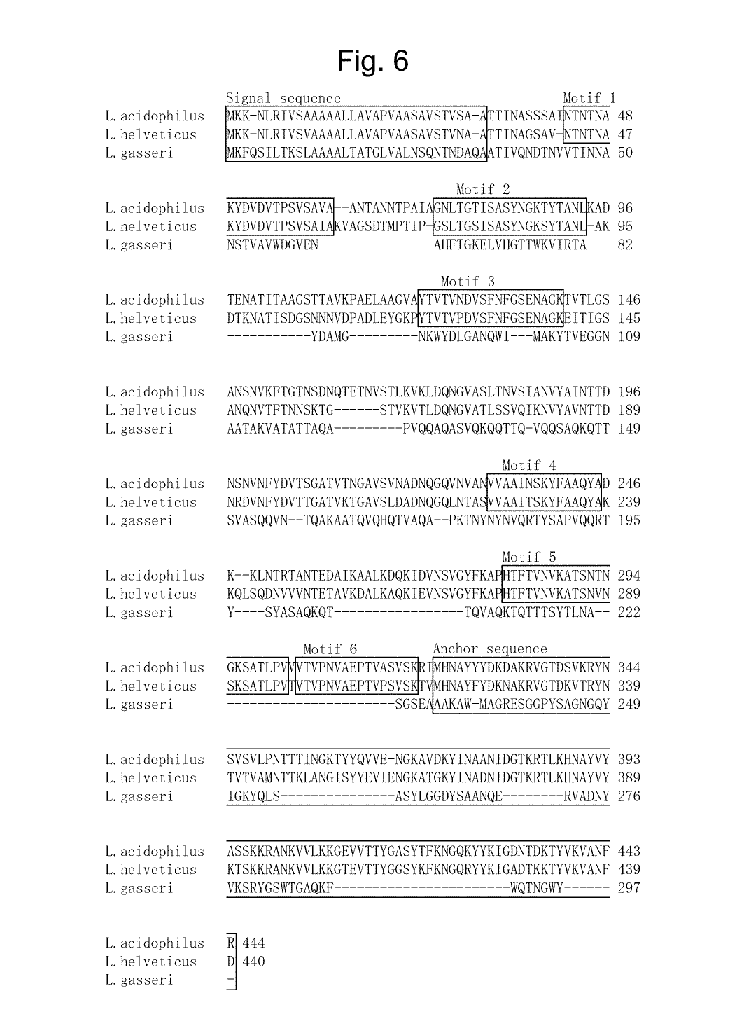

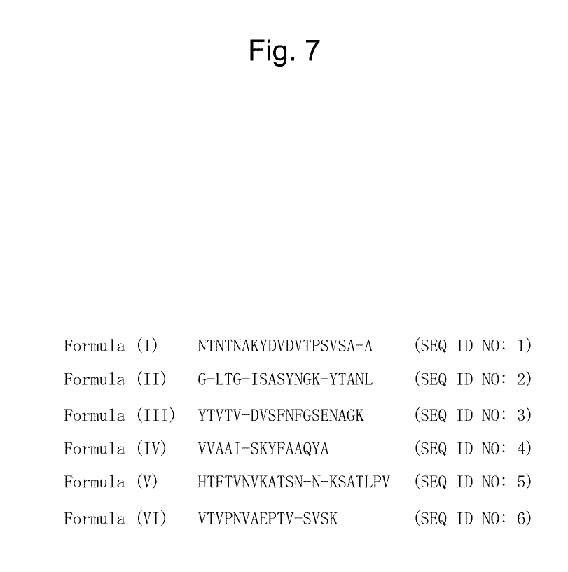

FIG. 6 shows the results of alignment analysis. The sequences of formulae (I) to (VI) were identified as sequences found in common in Lactobacillus acidophilus and Lactobacillus helveticus, both of which exhibit a high degree of binding to Umod, but not in Lactobacillus gasseri, which exhibits a low degree of binding to Umod (FIG. 7).

Further, in addition to Lactobacillus acidophilus and Lactobacillus helveticus, the following possibly related species of Lactobacillus acidophilus were selected for analysis from among other bacterial species of the genus Lactobacillus, the genome information of which was available: Lactobacillus crispatus (gi|113967820|gb|ABI49168.1|SlpB [Lactobacillus crispatus]), Lactobacillus amylovorus (gi|385816784|ref|YP_005853174.1|S-layer protein [Lactobacillus amylovorus GRL1118]), and Lactobacillus gallinarum (gi|51242255|gb|AAT99079.1|LgsF [Lactobacillus gallinarum]).

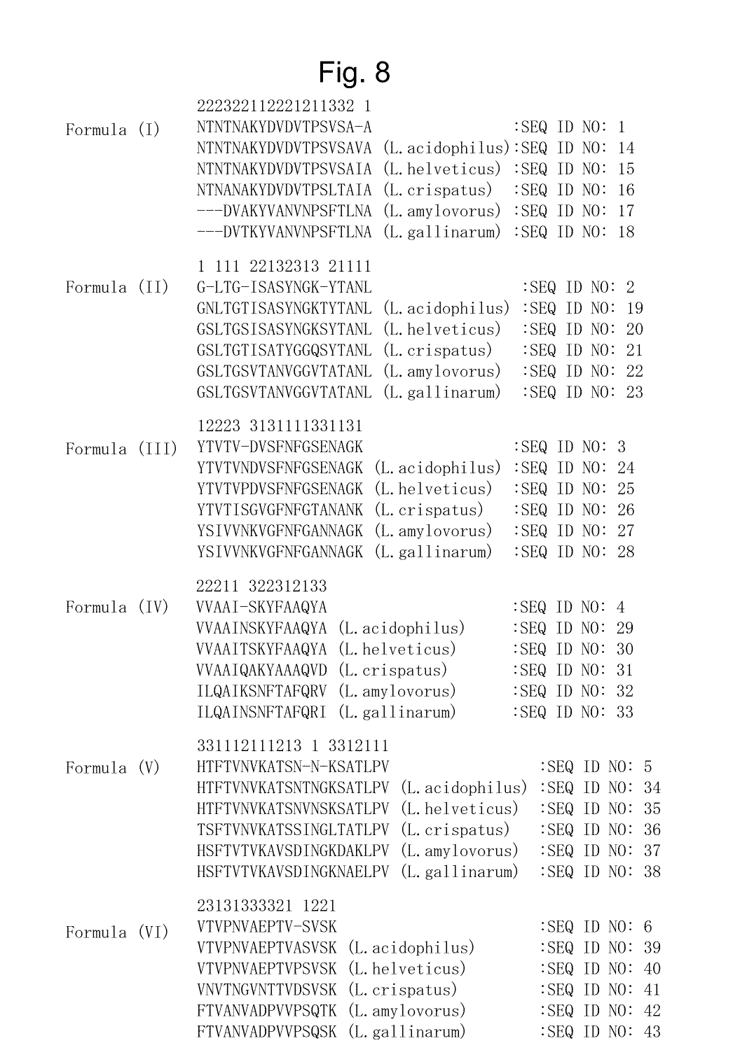

FIG. 8 shows the amino acid sequences of formulae (I) to (VI) and their corresponding partial amino acid sequences (SEQ ID NO: 14 to 43) of Lactobacillus acidophilus, Lactobacillus helveticus, Lactobacillus crispatus, Lactobacillus amylovorus, and Lactobacillus gallinarum. It was found that among these five different species, the amino acid sequence of formula (V) showed the highest degree of commonality while the amino acid sequence of formula (VI) showed the lowest degree of commonality.

### INDUSTRIAL APPLICABILITY

The present invention can be applied to the field of production of beverage or food products containing probiotics and pharmaceutical products such as mucosal vaccines.

All publications, patents, and patent applications cited herein are incorporated herein by reference in their entirety.

1. An agent for promoting substance incorporation in the intestinal tract, comprising a surface layer protein of a lactic acid bacterium of the genus Lactobacillus that comprises at least one of the peptide motifs consisting of amino acid sequences of the following formulae (I) to (VI) and has activity of binding to uromodulin (Umod) protein, or a fragment of the surface layer protein:
formula (I):
Asn-Thr-Asn-Thr-Asn-Ala-Lys-Tyr-Asp-Val-Asp-Val-
Thr-Pro-Ser-Val-Ser-Ala-X1-Ala (where X1
represents Val or Ile);
formula (II):
Gly-X2-Leu-Thr-Gly-X3-Ile-Ser-Ala-Ser-Tyr-Asn-
Gly-Lys-X4-Tyr-Thr-Ala-Asn-Leu (where X2
represents Asn or Ser, X3 represents Thr or
Ser, and X4 represents Thr or Ser);
formula (III):
Tyr-Thr-Val-Thr-Val-X5-Asp-Val-Ser-Phe-Asn-Phe-
Gly-Ser-Glu-Asn-Ala-Gly-Lys (where X5
represents Asn or Pro);
formula (IV):
Val-Val-Ala-Ala-Ile-X6-Ser-Lys-Tyr-Phe-Ala-Ala-
Gln-Tyr-Ala (where X6 represents Asn or Thr);
formula (V):
His-Thr-Phe-Thr-Val-Asn-Val-Lys-Ala-Thr-Ser-Asn-
X7-Asn-X8-Lys-Ser-Ala-Thr-Leu-Pro-Val (where X7
represents Thr or Val and X8 represents Gly or
Ser);
and
formula (VI):
Val-Thr-Val-Pro-Asn-Val-Ala-Glu-Pro-Thr-Val-X9-
Ser-Val-Ser-Lys (where X9 represents Ala or Pro).

2. The agent for promoting substance incorporation in the intestinal tract according to claim 1, comprising a surface layer protein of a lactic acid bacterium of the genus Lactobacillus that comprises at least one peptide motif consisting of an amino acid sequence including a deletion, substitution, or addition of 1 to 10 amino acids with respect to any one of the amino acid sequences of formulae (I) to (VI) and has activity of binding to uromodulin (Umod) protein.

3. The agent for promoting substance incorporation in the intestinal tract according to claim 1, wherein the surface layer protein is an S-layer protein from Lactobacillus acidophilus, Lactobacillus helveticus, Lactobacillus crispatus, Lactobacillus amylovorus, or Lactobacillus gallinarum.

4. The agent for promoting substance incorporation in the intestinal tract according to any one of claims 1, wherein the surface layer protein is SlpA protein from Lactobacillus acidophilus.

5. The agent for promoting substance incorporation in the intestinal tract according to claim 4, wherein SlpA protein from Lactobacillus acidophilus is a protein selected from the group consisting of the following (a) to (c):
(a) a protein consisting of the amino acid sequence of SEQ ID NO: 7;
(b) a protein consisting of an amino acid sequence including a deletion, substitution, or addition of one to several amino acids with respect to the amino acid sequence of SEQ ID NO: 7; and
(c) a protein having a sequence identity of 90% or more with the amino acid sequence of SEQ ID NO: 7.

6. A complex of the agent for promoting substance incorporation in the intestinal tract according to claim 1 and a substance to be incorporated into the intestinal tract.

7. The complex according to claim 6, wherein the substance to be incorporated into the intestinal tract is a food component.

8. The complex according to claim 7, wherein the food component is a lactic acid bacterium.

9. The complex according to claim 6, wherein the substance to be incorporated into the intestinal tract is a pharmaceutical component.

10. The complex according to claim 9, wherein the pharmaceutical component is a mucosal vaccine antigen.

11. A composition comprising the complex according to claim 6.

12. The composition according to claim 11, which is a beverage or food product, a pharmaceutical product, or an animal feed.

13. A method for screening for a lactic acid bacterium having a high level of ability to be transferred into the intestinal tract, comprising determining the level of expression of a protein comprising at least one of the peptide motifs consisting of the amino acid sequences of the following formulae (I) to (VI) in the surface layers of bacterial cells of a test lactic acid bacterium:
formula (I):
Asn-Thr-Asn-Thr-Asn-Ala-Lys-Tyr-Asp-Val-Asp-Val-
Thr-Pro-Ser-Val-Ser-Ala-X1-Ala (where X1
represents Val or Ile);
formula (II):
Gly-X2-Leu-Thr-Gly-X3-Ile-Ser-Ala-Ser-Tyr-Asn-
Gly-Lys-X4-Tyr-Thr-Ala-Asn-Leu (where X2
represents Asn or Ser, X3 represents Thr or
Ser, and X4 represents Thr or Ser);
formula (III):
Tyr-Thr-Val-Thr-Val-X5-Asp-Val-Ser-Phe-Asn-
Phe-Gly-Ser-Glu-Asn-Ala-Gly-Lys (where X5
represents Asn or Pro);
formula (IV):
Val-Val-Ala-Ala-Ile-X6-Ser-Lys-Tyr-Phe-Ala-
Ala-Gln-Tyr-Ala (where X6 represents Asn or
Thr);
formula (V):
His-Thr-Phe-Thr-Val-Asn-Val-Lys-Ala-Thr-Ser-
Asn-X7-Asn-X8-Lys-Ser-Ala-Thr-Leu-Pro-Val
(where X7 represents Thr or Val and X8
represents Gly or Ser);
and
formula (VI):
Val-Thr-Val-Pro-Asn-Val-Ala-Glu-Pro-Thr-Val-X9-
Ser-Val-Ser-Lys (where X9 represents Ala or Pro).
